(19) United States
(12) Patent Application Publication
(10) Pub. No.: US 2019/0021582 A1
(43) Pub. Date: Jan. 24, 2019

(54) IMAGING UNIT AND ENDOSCOPE

(71) Applicant: OLYMPUS CORPORATION, Tokyo (JP)

(72) Inventor: Toshiyuki SHIMIZU, Tokyo (JP)

(73) Assignee: OLYMPUS CORPORATION, Tokyo (JP)

(21) Appl. No.: 16/127,326

(22) Filed: Sep. 11, 2018

Related U.S. Application Data
(63) Continuation of application No. PCT/JP2017/017363, filed on May 8, 2017.

(30) Foreign Application Priority Data
May 19, 2016 (JP) 2016-100291

(51) Int. Cl. A61B 1/05; A61B 1/00; H01L 27/146

(52) U.S. Cl. CPC A61B 1/051; H01L 27/14618; A61B 1/0011

(57) ABSTRACT

An imaging unit includes: a semiconductor package that includes a light receiver of an imaging element formed on a front surface of the semiconductor package, and a sensor electrode formed on a back surface of the semiconductor package; a circuit board that includes a connection electrode electrically and mechanically connected to the sensor electrode through a bump on a front surface of the circuit board; an enclosing member configured to enclose the semiconductor package; a first filler that is filled in space enclosed by the enclosing member; and a second filler that is filled on a joint surface between the semiconductor package and the circuit board, and that has a smaller linear expansion per unit length at sterilization temperature than that of the first filler.

### CROSS REFERENCES TO RELATED APPLICATIONS

This application is a continuation of PCT international application Ser. No. PCT/JP2017/017363 filed on May 8, 2017 which designates the United States, incorporated herein by reference, and which claims the benefit of priority from Japanese Patent Applications No. 2016-100291, filed on May 19, 2016, incorporated herein by reference.

### BACKGROUND

### 1. Technical Field

The present disclosure relates to an imaging unit that is arranged at a distal end of an insertion portion of an endoscope inserted into a body of a subject to image the interior of the body, and the endoscope.

### 2. Related Art

Endoscopes have been widely used for various kinds of inspections in a medical field and an industrial field conventionally. Among these, a medical endoscope apparatus can acquire an in-vivo image of a body cavity without incising a subject, by inserting a flexible elongated insertion portion with an imaging element provided at its distal end into a body cavity of the subject such as a patient and, further, can perform treatment procedures by projecting a treatment instrument out from an end of the insertion portion as necessary, and is, therefore, used generally.

At a distal end of the insertion portion of such an endoscope apparatus, an imaging unit that includes an imaging element, a circuit board on which electronic components, such as a capacitor, an IC chip, and the like constituting a driving circuit of the imaging element, and cables are mounted, and an electric connector member, such as a TAB tape connecting the imaging element and the circuit board is embedded, and a filler is filled around the imaging element and the electronic components to protect them.

For endoscopes used for medical purposes, autoclave sterilization (115° C. to 138° C., atmospheric pressure approximately +0.2 MPa) is performed for sterilization. Because the TAB tape or joint portions of the electronic components can be damaged by expansion of the filler when heated to the sterilization temperature, an imaging unit that uses two kinds of sealing resins having different linear expansion coefficients has been suggested (for example, refer to Japanese Patent No. 4578913).

### SUMMARY

In some embodiments, an imaging unit includes: a semiconductor package that includes a light receiver of an imaging element formed on a front surface of the semiconductor package, and a sensor electrode formed on a back surface of the semiconductor package; a circuit board that includes a connection electrode electrically and mechanically connected to the sensor electrode through a bump on a front surface of the circuit board; an enclosing member configured to enclose the semiconductor package; a first filler that is filled in space enclosed by the enclosing member; and a second filler that is filled on a joint surface between the semiconductor package and the circuit board, and that has a smaller linear expansion per unit length at sterilization temperature than that of the first filler.

In some embodiments, an endoscope includes an insertion portion in which the imaging unit according to the above-described imaging unit is arranged at a distal end of the insertion portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically showing an entire configuration of an endoscope system according to a first embodiment of the disclosure;

FIG. 2 is a cross-section of an imaging unit that is arranged at a distal end of an endoscope shown in FIG. 1;

FIG. 3 is a perspective view of the imaging unit shown in FIG. 2;

FIG. 4 is a diagram showing a relationship between temperature and linear expansion of a first filler and a second filler used in the first embodiment of the disclosure;

FIG. 5 is a cross-section of an imaging unit according to a second embodiment of the disclosure;

FIG. 6 is a perspective view of the imaging unit shown in FIG. 5;

FIG. 7 is a cross-section of an imaging unit according to a third embodiment of the disclosure;

FIG. 8 is a perspective view of the imaging unit shown in FIG. 7;

FIG. 9 is a side view of the imaging unit shown in FIG. 7;

FIG. 10 is a cross-section of an imaging unit according to a fourth embodiment of the disclosure;

FIG. 11 is a perspective view of the imaging unit shown in FIG. 10 from bottom;

FIG. 12 is a perspective view of the imaging unit shown in FIG. 10 from above; and

FIG. 13 is a diagram for explaining a positional relationship of a recessed portion of a circuit board and a sensor electrode shown in FIG. 10.

### DETAILED DESCRIPTION

In the following explanation, as forms to implement the disclosure (hereinafter, “embodiments”), an endoscope system equipped with an imaging unit is explained. The embodiments are not intended to limit the disclosure. Furthermore, like reference symbols are assigned to like parts throughout the drawings. Still further, the drawings are typical examples, and it should be noted that a relationship between thickness and width of respective parts, a ratio of the respective parts, and the like differ from an actual situation. Moreover, also among the drawings, dimensions and ratios may include differences.

### First Embodiment

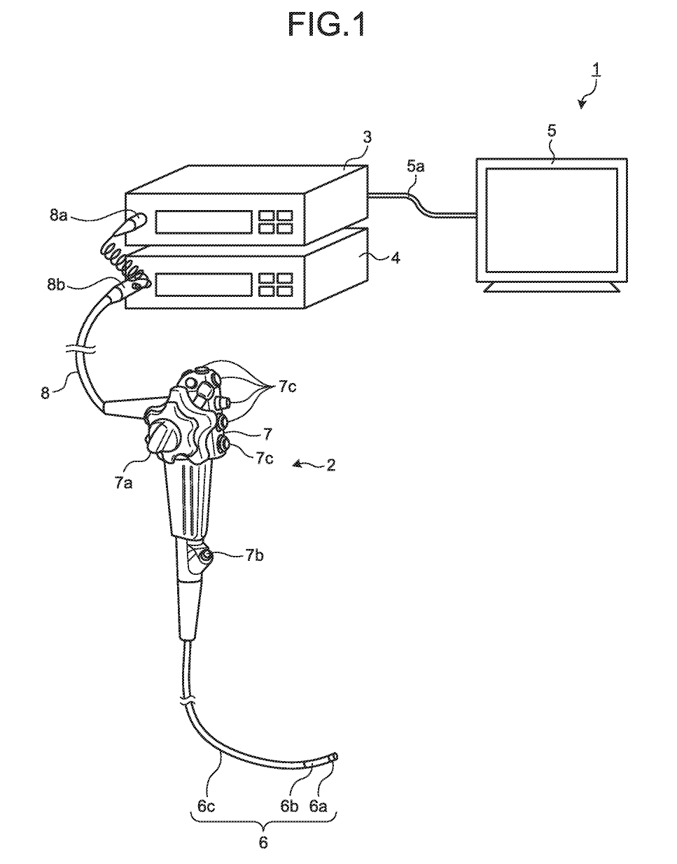

FIG. 1 is a diagram schematically showing an entire configuration of an endoscope system according to a first embodiment of the disclosure. As shown in FIG. 1, an endoscope system 1 according to the first embodiment includes an endoscope 2 that is introduced into a body of a subject and images an interior of the subject to generate an image signal of the interior of the subject, an information processing device 3 that subjects the image signal acquired by the endoscope 2 to predetermined image processing and that controls respective parts of the endoscope system 1, a light source device 4 that generates illumination light of the endoscope 2, and a display device 5 that displays an image of the image signal subjected to the image processing by the information processing device 3.

The endoscope 2 includes an insertion portion 6 that is inserted into the body of the subject, an operating unit 7 that is arranged on a proximal end side of the insertion portion 6 held by an operator, and a flexible universal cord 8 that extends from the operating unit 7.

The insertion portion 6 is implemented by using an illumination fiber (light guide cable), an electric cable, an optical fiber, and the like. The insertion portion 6 has an end portion 6a in which an imaging unit described later is arranged, a bend portion 6b that is flexibly bendable and is constituted of multiple bending pieces, a flexible tube portion 6c that has flexibility and is arranged on a proximal end side of the bend portion 6b. At the end portion 6a, an illuminating unit that illuminates the interior of the body of the subject through an illumination lens, an observing unit that images the interior of the body of the subject, an opening portion that communicates with a channel for treatment equipment, and an air and water supply nozzle (not shown) are arranged.

The operating unit 7 has a bending knob 7a to bend the bend portion 6b in a vertical direction and a horizontal direction, a treatment-equipment insertion portion 7b to which a treatment equipment, such as a bio-forceps and a laser scalpel, is inserted, the information processing device 3, the light source device 4, and a plurality of switch units 7c to operate peripheries, such as an air supply device, a water supply device, and a gas supply device. Treatment equipment inserted from the treatment-equipment insertion portion 7b comes out from the opening portion at the end of the insertion portion 6 through the channel for treatment equipment arranged inside.

The universal cord 8 is constructed of an illumination fiber, a cable, or the like. The universal cord 8 branches off at a proximal end, and one branched end is a connector 8a, and the other proximal end is a connector 8b. The connector 8a is detachable to a connector of the information processing device 3. The connector 8b is detachable to the light source device 4. The universal cord 8 propagates illumination light emitted from the light source device 4 to the end portion 6a through the connector 8b and the illumination fiber. Moreover, the universal cord 8 transmits an image signal acquired by the imaging unit described later to the information processing device 3 through the cable and the connector 8a.

The information processing device 3 subjects an image signal output from the connector 8a to predetermined image processing, and controls the entire endoscope system 1.

The light source device 4 is constituted of a light source that emits light, a condenser lens, and the like. The light source device 4 emits light from the light source under control of the information processing device 3, to provide it to the endoscope 2 connected through the connector 8b and the illumination fiber of the universal cord 8 as illumination light for the interior of the body of the subject to be imaged.

The display device 5 is constructed of a liquid crystal or an organic EL (electro luminescence) display or the like. The display device 5 displays various kinds of information including the image subjected to the predetermined image processing by the information processing device 3 through an image cable 5a. Thus, an operator operates the endoscope 2 viewing an image (in-vivo image) displayed by the display device 5, and thereby can observe a desirable position inside the body of the subject and determine a condition.

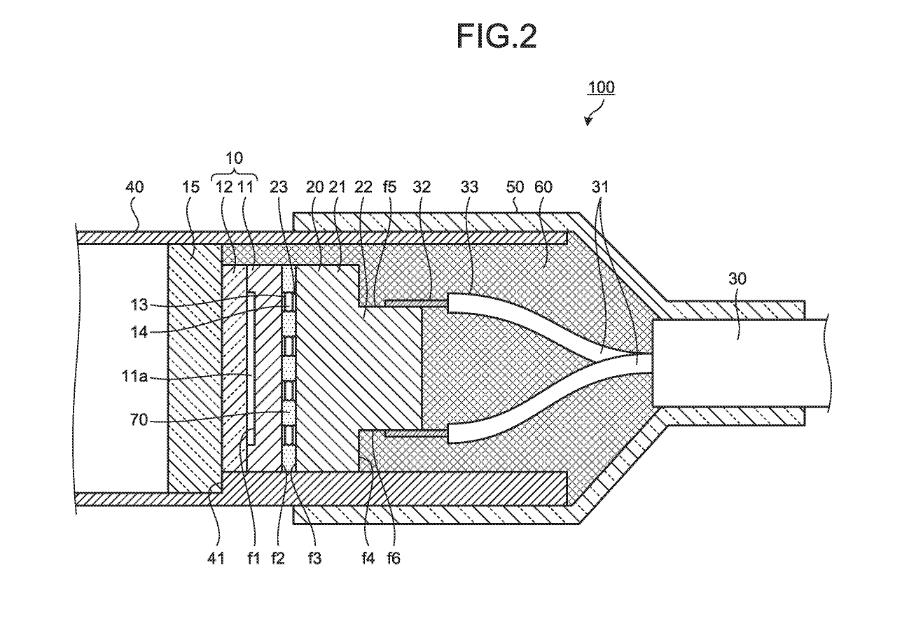

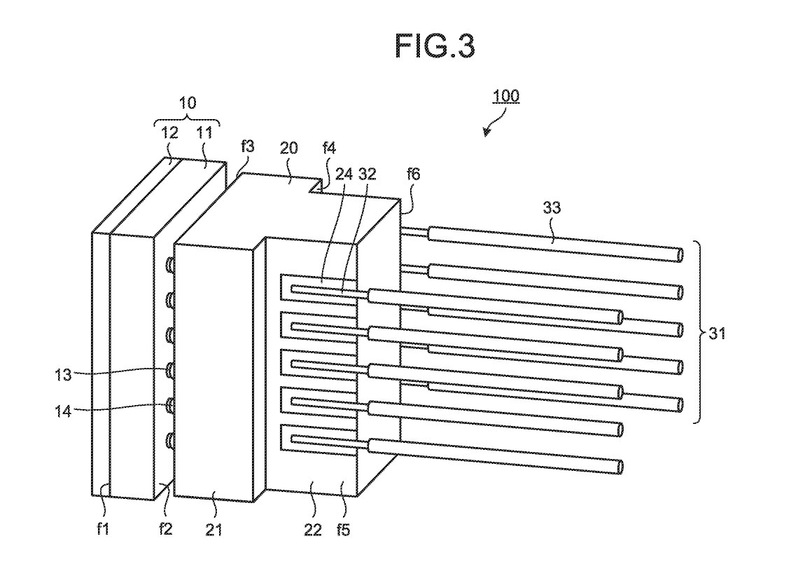

Next, details of the imaging unit used in the endoscope system 1 are explained. FIG. 2 is a cross-section of the imaging unit that is arranged at a distal end of the endoscope shown in FIG. 1. FIG. 3 is a perspective view of the imaging unit shown in FIG. 2. In FIG. 3, illustration of a holding frame 40, a heat-shrinkable tube 50, a first filler 60, a second filler 70, and a centered cover glass 15 of an imaging unit 100 in FIG. 2 is omitted, and a 90° rotated state is shown such that a side surface f5 of a circuit board 20 faces forward.

The imaging unit 100 includes: a semiconductor package 10 which includes an imaging element 11 and in which a sensor electrode 13 is formed on an f2 surface that is a back surface of the 100 of the semiconductor package 10; the circuit board 20 including a main body 21 in which a connection electrode 23 is formed, and an attaching portion 22 that protrudes from a back surface of the main body 21; a stranded cable 30 in which a plurality of signal cables are stranded; the holding frame 40 that holds the semiconductor package 10; the heat-shrinkable tube 50 that is a covering member that covers a proximal end portion of the holding frame 40; a first filler 60 that is filled in space enclosed by the holding frame 40 and the heat-shrinkable tube 50; and a second filler 70 that is filled on a joint surface between the semiconductor package 10 and the circuit board 20. In the first embodiment, the holding frame 40 and the heat-shrinkable tube 50 function as an enclosing member. The enclosing member is constituted of the holding frame 40 and the heat-shrinkable tube 50 in the first embodiment, but is not limited to this structure. For example, it can have a structure including another member combined, or have a structure only with the heat-shrinkable tube 50.

In the semiconductor package 10, a cover glass 12 is affixed to protect a light receiving portion 11a of the imaging element 11, and the centered cover glass 15 having a larger diameter than the semiconductor package 10 is affixed to the cover glass 12 on a distal end side of the cover glass 12. The semiconductor package 10 is held by the holding frame 40 as a periphery portion of the centered cover glass 15 that is not in contact with the semiconductor package 10 abuts on a positioning portion 41 of the holding frame 40. Light gathered by the lens unit enters an f1 surface (light receiving surface) of the imaging element 11 through the centered cover glass 15 and the cover glass 12. On the f2 surface of the imaging element 11, the sensor electrode 13 and a bump 14 formed with solder or the like are formed. The semiconductor package 10 is preferable to be a CSP (chip size package) that is fabricated by subjecting an imaging element chip in a state of wafer to wiring, electrode formation, resin filling, and dicing to be finally formed into the semiconductor package in a size remaining in the size of the imaging element chip as it is.

The circuit board 20 includes the main body 21 having the connection electrode 23 formed on a front surface of the main body 21, and the attaching portion 22 that protrudes from the back surface of the main body 21, and that has cable connection electrodes 24 formed on two opposing side surfaces f5 and f6 out of side surfaces of the protrusion. The main body 21 and the attaching portion 22 can be a board formed in one piece, or can be one obtained by combining individually fabricated boards. The circuit board 20 has a planar shape in which a plurality of substrates with wirings formed thereon are layered (multiple substrates parallel to a front surface f3 and a back surface f4 are layered). For a substrate to be layered, a ceramic substrate, a glass epoxy substrate, a flexible substrate, a glass substrate, a silicone substrate, or the like is used. Inside the circuit board 20, a plurality of vias (not shown) for conduction of wirings on the layered substrates are formed.

On the front surface f3 of the main body 21 of the circuit board 20, the connection electrode 23 is formed and is electrically and mechanically connected to the sensor electrode 13 of the semiconductor package 10 through the bump 14.

The stranded cable 30 is constituted of signal cables 31, which are 10 solid wire cables, and an outer periphery of the signal cables 31 is covered with an overall shield and an overall sheath. At a distal end portion of the stranded cable 30, the overall shield and the overall sheath are removed. Furthermore, the signal cable 31 has a core line 32 and an outer cover 33 arranged around an outer periphery of the core line 32. The outer cover 33 is removed at the distal end portion of the signal cable 31 such that the core line 32 is gradually exposed from the distal end portion. In the first embodiment, the core lines 32 of the signal cables 31 are electrically and mechanically connected to the cable connection electrodes 24 that are formed on the opposing side surfaces f5 and f6 of the attaching portion 22 of the circuit board 20 through solder or the like not shown. Moreover, in the first embodiment, the circuit board 20 and the signal cables 31 (stranded cable 30) that are connected to the cable connection electrodes 24 on the side surface f5 and the side surface f6 of the circuit board 20 fit within a size of a surface of projection of the semiconductor package 10 in an optical axis of the semiconductor package 10. Thus, downsizing in diameter of the imaging unit 100 is enabled.

The heat-shrinkable tube 50 covers the proximal end portion of the holding frame 40 and the distal end of the stranded cable 30, and is intimately fixed to the holding frame 40 and the overall sheath of the stranded cable 30. In space enclosed by the holding frame 40 and the heat-shrinkable tube 50, the insulative first filler 60 is filled. The first filler 60 is made from a material highly resistant to moisture, and an influence of humidity to the semiconductor package 10 can be reduced.

The joint surface between the semiconductor package 10 and the circuit board 20, that is, a joint portion between the connection electrode 23 and the sensor electrode 13 is sealed with the insulative second filler 70. By sealing the joint surface between the semiconductor package 10 and the circuit board 20 with the second filler 70, the bond strength can be improved. The second filler 70 has a smaller linear expansion per unit length than that of the first filler 60 when heated from room temperature to sterilization temperature. By filling the second filler 70 having the smaller linear expansion per unit length at the sterilization temperature than that of the first filler 60 on the joint surface between the semiconductor package 10 and the circuit board 20, a contact area of which is small, and for which reliable connection is necessary, an influence of thermal expansion at the time of sterilization processing to the joint portion can be reduced.

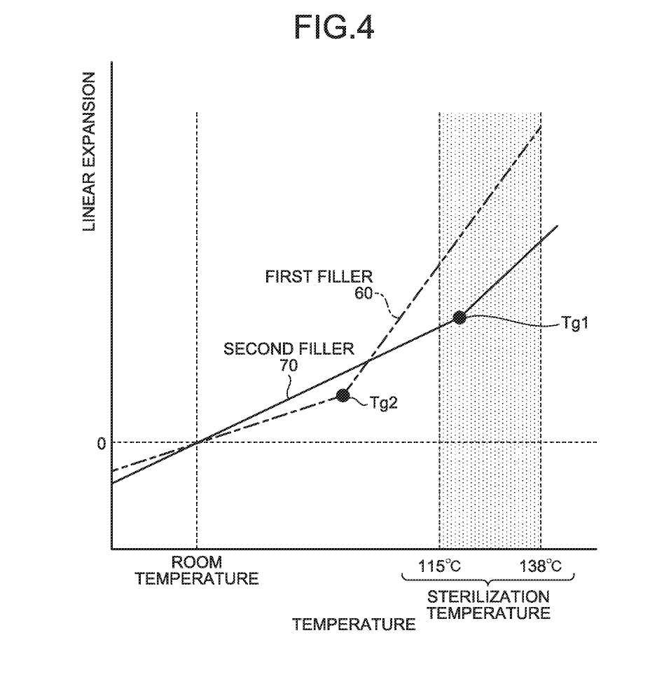

The linear expansion per unit length at the sterilization temperature is linear expansion per unit length when heated to the sterilization temperature (115° C. to 138° C.) when the linear expansion at room temperature is 0 as shown in FIG. 4. As shown in FIG. 4, a linear expansion rate changes at glass transition points Tg1, Tg2. Accordingly, it is preferable that a filler be selected based on not the linear expansion rate, but linear expansion per unit length from room temperature to sterilization temperature. As the second filler 70, by selecting one having a smaller linear expansion per unit length from room temperature to sterilization temperature than that of the first filler 60, an influence of thermal expansion at the time of sterilization processing to the joint portion can be effectively reduced.

Furthermore, it is preferable that the viscosity of the second filler 70 before hardening be smaller than the viscosity of the first filler 60 before hardening. This makes it easy to fill the second filler 70 on the joint surface between the semiconductor package 10 and the circuit board 20.

The imaging unit 100 according to the first embodiment fills the second filler 70 having a smaller linear expansion per unit length when heated from room temperature to sterilization temperature than that of the first filler 60 on the joint surface between the semiconductor package 10 and the circuit board 20 and, therefore, can reduce an influence of thermal expansion at the time of sterilization processing to the joint portion. Moreover, by using a material highly resistant to moisture as the first filler 60, an influence of humidity to the semiconductor package 10 can be reduced. Furthermore, the circuit board 20 and the signal cables 31 (the stranded cable 30) respectively connected to the cable connection electrodes 24 on the side surface f5 and the side surface f6 of the circuit board 20 fit within a size of the surface of projection of the semiconductor package 10 in the optical axis direction of the semiconductor package 10 and, therefore, downsizing in diameter of the imaging unit 100 is possible.

### Second Embodiment

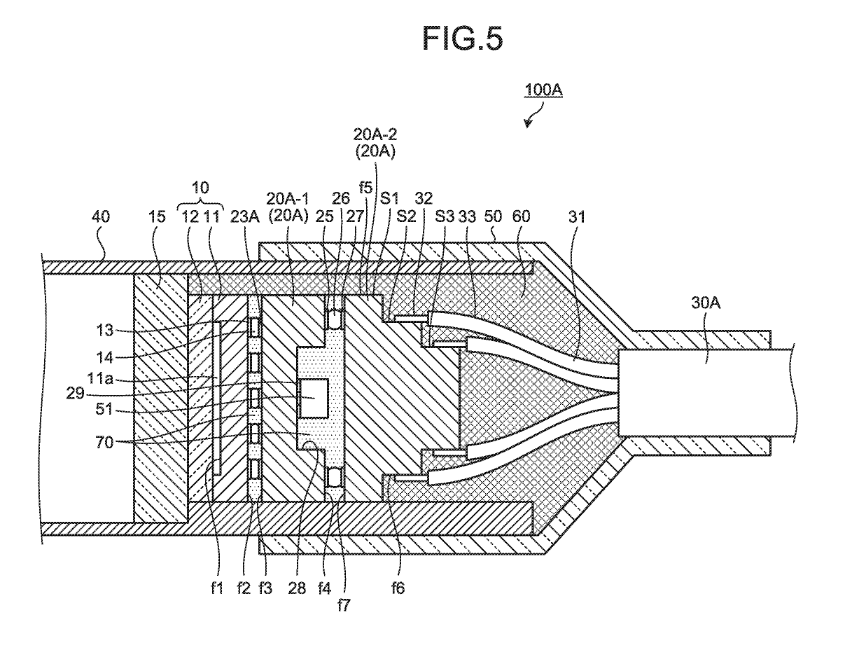

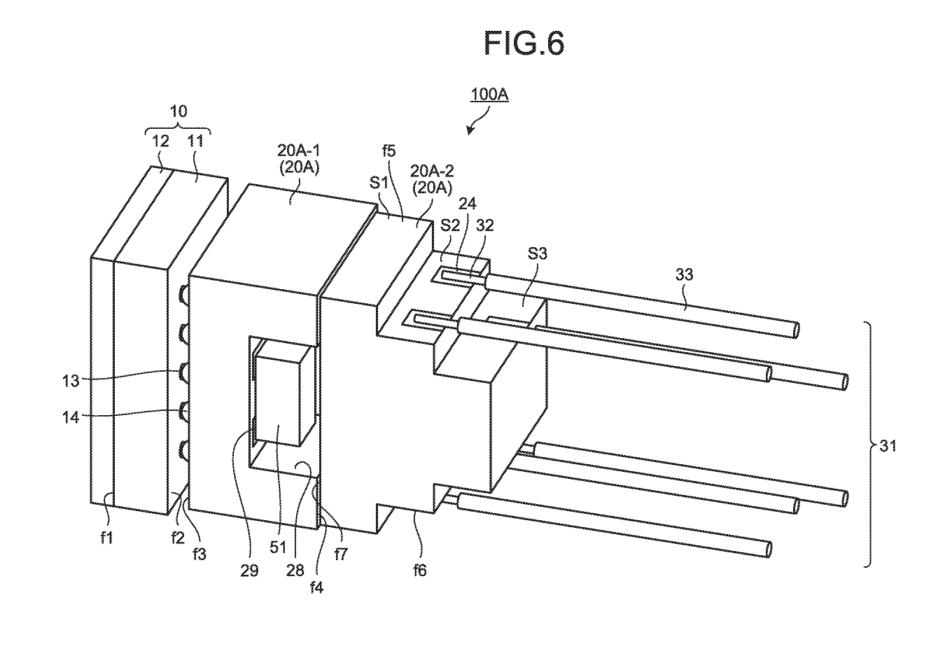

FIG. 5 is a cross-section of an imaging unit according to a second embodiment of the disclosure. FIG. 6 is a perspective view of the imaging unit shown in FIG. 5. In FIG. 6, illustration of the holding frame 40, the heat-shrinkable tube 50, the first filler 60, the second filler 70, and the centered cover glass 15 of an imaging unit 100A in FIG. 5 is omitted.

In the imaging unit 100A according to the second embodiment, a circuit board 20A includes a first board 20A-1 and a second board 20A-2. The first board 20A-1 has a first connection electrode 23A and a second connection electrode 25 formed on the front surface f3 and the back surface f4, respectively. The first connection electrode 23A on the front surface f3 is electrically and mechanically connected to the sensor electrode 13 of the semiconductor package 10 through the bump 14. The second board 20A-2 has a third connection electrode 27 formed on a front surface f7, and the cable connection electrodes 24 formed on the side surface f5 and the side surface f6. The third connection electrode 27 is electrically and mechanically connected to the second connection electrode 25 of the first board 20A-1 through a bump 26. The bump 26 can be a solder ball, a metal-core solder ball, a resin core ball, a gold bump, and the like.

In the back surface f4 of the first board 20A-1, a recessed portion 28 is arranged, and an electronic component 51 is mounted in a mounting land 29 formed in the recessed portion 28 through a conductive member, such as solder. Around a joint portion between the electronic component 51 and the mounting land 29, that is, between a bottom surface of the electronic component 51 and the recessed portion 28, the second filler 70 is filled. Moreover, between the back surface f4 of the first board 20A-1 and the front surface f7 of the second board 20A-2, and in the recessed portion 28, the second filler 70 is filled. The recessed portion 28 is formed in the first board 20A-1 in the second embodiment, but the recessed portion 28 can be formed in the second board 20A-2.

The second board 20A-2 has step portions S1, S2, and S3 at the opposing side surfaces f5 and f6. The surface f5 and the side surface f6 has the step portions S1 to S3 so as to come close to each other on the proximal end side in the optical axis direction of the semiconductor package 10. At the step portions S2 and S3, the cable connection electrodes 24 are respectively arranged, and the core lines of the signal cables 31 are electrically and mechanically connected to the cable connection electrodes 24.

Furthermore, the imaging unit 100A is structured such that the first board 20A-1, the second board 20A-2, and the signal cables 31 (stranded cable 30A) connected to the cable connection electrodes 24 fit in the size of a surface of projection of the semiconductor package 10 in the optical direction of the semiconductor package 10. This enables downsizing in diameter of the imaging unit 100A.

In the imaging unit 100A according to the second embodiment, the second filler 70 having a smaller linear expansion per unit length when heated from room temperature to sterilization temperature than that of the first filler 60 is filled on the joint surface between the semiconductor package 10 and the first board 20A-1, between the f4 surface of the first board 20A-1 and the f7 surface of the second board 20A-2, and in the recessed portion 28. Therefore, an influence of thermal expansion at the time of sterilization processing can be reduced. Moreover, by using a material highly resistant to moisture is used as the first filler 60, an influence of humidity to the semiconductor package 10 can be reduced.

### Third Embodiment

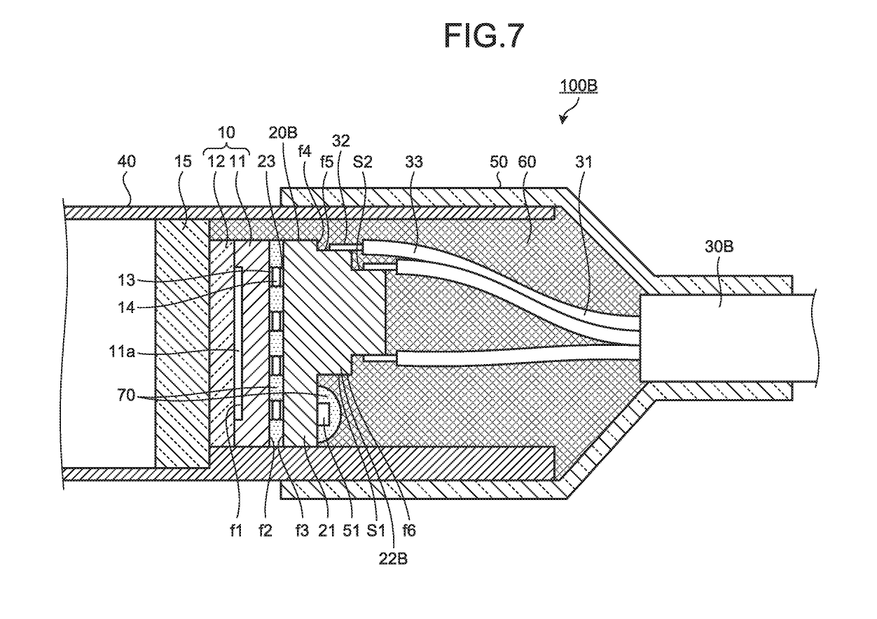

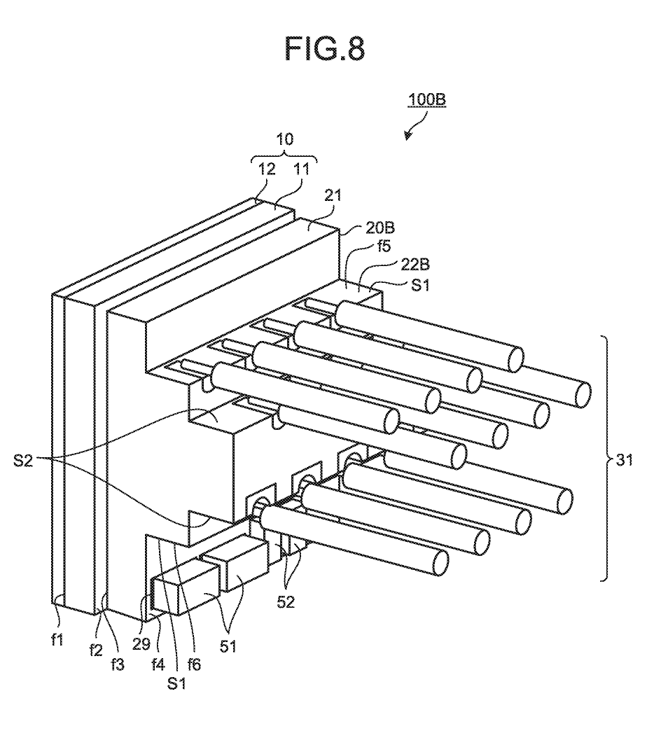

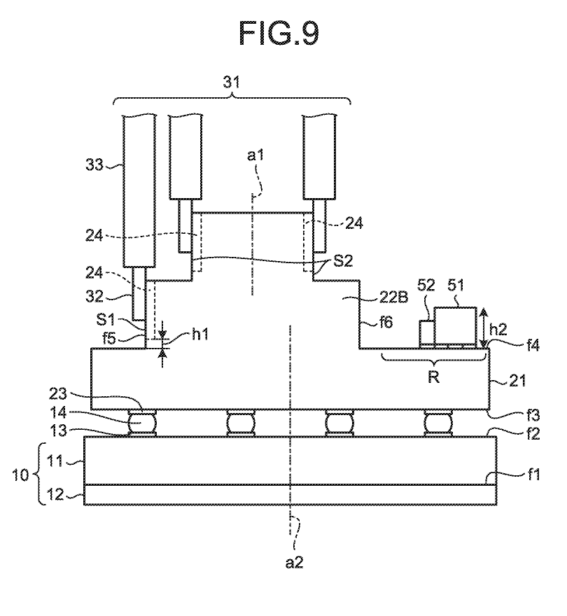

FIG. 7 is a cross-section of an imaging unit according to a third embodiment of the disclosure. FIG. 8 is a perspective view of the imaging unit shown in FIG. 7. FIG. 9 is a side view of the imaging unit shown in FIG. 7. In FIG. 8 and FIG. 9, illustration of the holding frame 40, the heat-shrinkable tube 50, the first filler 60, the second filler 70, and the centered cover glass 15 of an imaging unit 100B is omitted.

In an imaging unit 100B according to the third embodiment, an electronic-component mounting area in which electronic components 51 and 52 are mounted is arranged on the back surface f4 of a circuit board 20B, and an attaching portion 22B to which the signal cables 31 of the circuit board 20B are connected protrudes from the main body 21 such that the center plane of the opposing side surface f5 and side surface f6 on which the cable connection electrodes 24 are formed is shifted in position from the center plane of the semiconductor package 10.

The attaching portion 22B protrudes from the main body 21 in steps, and the step portions S1 and S2 are arranged on the opposing side surfaces f5 and f6. The step portions S1 and S2 are arranged so as to come close to each other on the proximal end side in the optical axis direction of the semiconductor package 10. On the step portion S1 and S2 on a side of the side surface f5 and on the step portion S2 on a side of the side surface f6, the cable connection electrodes 24 are arranged, and the core lines 32 of the signal cables 31 are electrically and mechanically connected to the cable connection electrodes 24.

As shown in FIG. 8, the cable connection electrodes 24 on the side of the side surface f5 are formed such that the cable connection electrodes 24 formed on the step portion S1 and on the step portion S2 are arranged in a houndstooth check (zigzag pattern). Moreover, the cable connection electrodes 24 formed facing each other on the step portions S2 of the side surface f5 and the side surface f6 are also arranged in a houndstooth check (zigzag pattern). By arranging the cable connection electrodes 24 in a houndstooth check (zigzag pattern), the packing density of the signal cables 31 can be improved.

The attaching portion 22B is formed in one piece integrated with the main body 21, as shown in FIG. 9, protruding from the main body 21 such that a center plane a1 of the side surface f5 and the side surface f6 on which the cable connection electrodes 24 are formed facing each other is shifted in position (shifted leftward in FIG. 9) from a center plane a2 of side surfaces of the semiconductor package 10 parallel to the side surface f5 and the side surface f6 of the attaching portion 22B. Thus, a part of the back surface f4 of the main body 21 on one side can be used as an electronic-component mounting area R. When the electronic components 51, 52 are mounted on the mounting land 29, solder is supplied to the mounting land 29 with a dispenser needle from the upper side in the drawing. In the third embodiment, because the electronic-component mounting area R is arranged beside the attaching portion 22B on one side of the back surface f4 of the main body 21, when supplying solder with the dispenser needle, the dispenser needle and the attaching portion 22B, particularly, the step portions S1, S2 do not interfere with each other, and solder can be accurately supplied from above, and the electronic components 51, 52 can be, therefore, mounted easily and precisely.

Furthermore, when the electronic components 51, 52 includes a capacitor (decoupling capacitor), the decoupling capacitor can be arranged closed to the imaging element 11 through the main body 21 adjacent to the imaging element 11. Therefore, an impedance between the imaging element 11 and the coupling capacitor can be reduced, and stable driving of the imaging element 11 and speedup of the imaging element 11 are possible.

Around the joint portion between the electronic components 51 and 52 and the mounting land 29, that is, between the bottom surfaces of the electronic components 51 and 52 and the back surface f4 of the main body 21, the second filler 70 is filled. Moreover, the periphery of the electronic components 51 and 52 are sealed with the second filler 70.

The cable connection electrodes 24 formed on the step portion S1 are arranged apart from the main body 21, and the cable connection electrodes 24 formed on the step portion S2 are arranged apart from the step portion S1. The cable connection electrodes 24 formed on the step portion S1 are arranged to overlap with the electronic components 51, 52 in the optical axis direction. Overlapping with the electronic components 51, 52 in the optical axis direction means that a distance h1 from an end of the cable connection electrode 24 on a side of the main body 21 to the main body 21 is shorter than a height h2 of the electronic component 51. By forming the cable connection electrodes 24 to be apart from the main body 21 or the step portion S1, a risk of short circuit caused by solder overflow, or the like can be reduced. Furthermore, by arranging the cable connection electrodes 24 formed on the step portion S1 to overlap with the electronic components 51, 52 in the optical axis direction, the length of the attaching portion 22B in the optical axis direction is shortened.

Moreover, the imaging unit 100B is structured such that the circuit board 20B, the electronic components 51 and 52, and the signal cables 31 (stranded cable 30B) connected to the respective cable connection electrodes 24 fit within the size of a surface of projection of the semiconductor package 10 in the optical axis direction of the semiconductor package 10. Thus, downsizing in diameter of the imaging unit 100B is enabled.

In the imaging unit 100B according to the third embodiment, the second filler 70 having a smaller linear expansion per unit length when heated from room temperature to sterilization temperature than that of the first filler 60 is filled on the joint surface between the semiconductor package 10 and the circuit board 20B, and around the joint portion of the electronic components 51 and 52 and the mounting land 29. Therefore, an influence of thermal expansion to the joint portion at the time of sterilization processing can be reduced. Moreover, by using a material highly resistant to moisture is used as the first filler 60, an influence of humidity to the semiconductor package 10 can be reduced.

In the third embodiment, the main body 21 has the step portions S1 and S2 on the opposing two side surfaces f5 and f6, but it is only required that at least one side, preferably the side surface on a shifted side (side surface f5 in the third embodiment) has the step portions S1, S2, and the cable connection electrodes 24 are arranged in the step portions S1 and S2.

### Fourth Embodiment

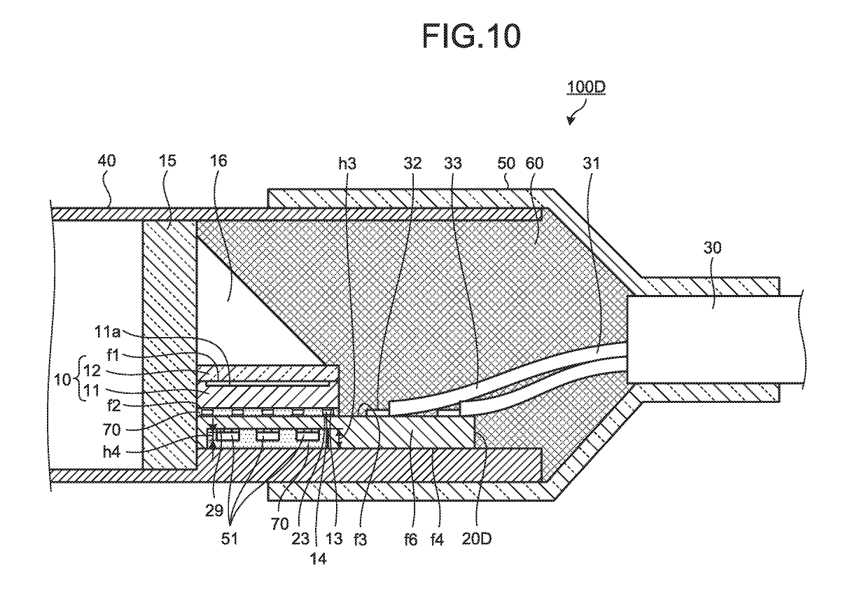

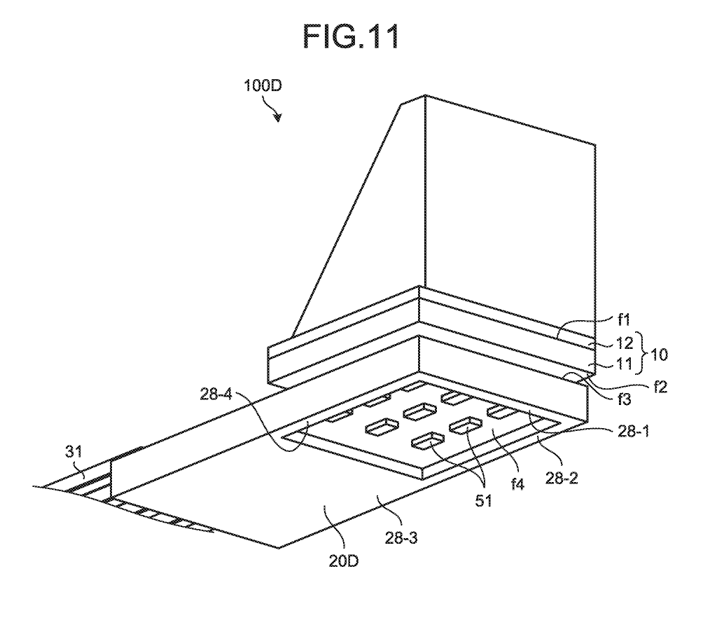

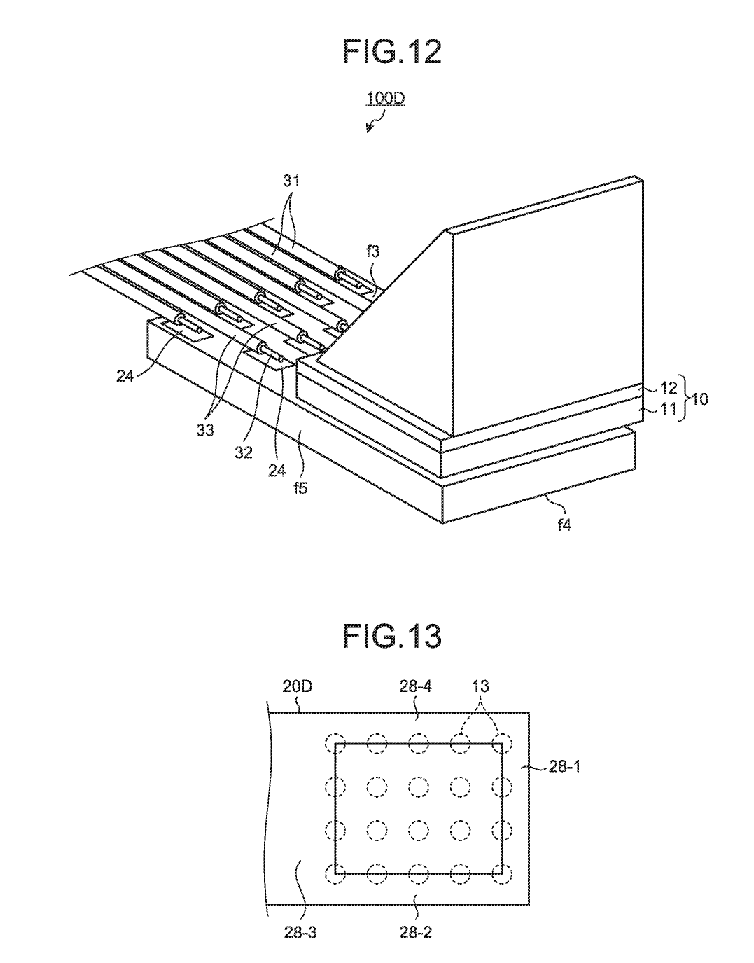

FIG. 10 is a cross-section of an imaging unit according to a fourth embodiment of the disclosure. FIG. 11 is a perspective view of the imaging unit shown in FIG. 10 from bottom. FIG. 12 is a perspective view of the imaging unit shown in FIG. 10 from above. FIG. 13 is a diagram for explaining a positional relationship of a recessed portion of a circuit board and a sensor electrode shown in FIG. 10. In FIG. 11 and FIG. 12, illustration of the holding frame 40, the heat-shrinkable tube 50, the first filler 60, the second filler 70, and the centered cover glass 15 of an imaging unit 100D in FIG. 10 is omitted.

The imaging unit 100D includes a prism 16 that collects and reflects incident light, and the imaging element 11 receives the light input from the prism 16. The semiconductor package 10 is so-called horizontal type in which the f1 surface being the light receiving surface of the imaging element 11 is arranged parallel to the optical axis direction.

A circuit board 20D has the connection electrodes 23 to which the sensor electrode 13 is connected, and the cable connection electrodes 24 to which the signal cables are connected. The connection electrodes 23 and the cable connection electrodes 24 are arranged side by side on the front surface f3. The circuit board 20D has wall portions 28-1, 28-2, 28-3, 28-4 formed on the entire perimeter of the back surface f4. When the circuit board 20D is made thin for downsizing the imaging unit 100D in diameter, warpage can occur in the circuit board 20D, and the reliability in connection between the semiconductor package 10 and the circuit board 20D can be deteriorated. However, by forming the wall portions 28-1, 28-2, 28-3, 28-4 on the back surface of the circuit board 20D, a warp of the circuit board 20D can be reduced.

On the front surface f3 of the circuit board 20D, the connection electrodes 23 to which the sensor electrodes 13 are connected, and the cable connection electrode 24 to which the signal cables 31 are connected are arranged, aligned in a direction in which the signal cables 31 extend (hereinafter, optical axis direction). The cable connection electrodes 24 are arranged in a houndstooth check (zigzag pattern) to decrease the diameter of the imaging unit 100D while improving the packing density of the signal cables 31.

In the circuit board 20D, the mounting land 29 is formed in an area in which the semiconductor package 10 of the back surface f4 is mounted, and the electronic component 51 is mounted in this mounting land 29 through a conductive member, such as solder. The area in which the mounting land 29 is formed is surrounded by the wall portions 28-1, 28-2, 28-3, 28-4. Around a joint portion of the electronic component 51 and the mounting land 29, that is, between the bottom surface of the electronic component 51 and the back surface f4 of the circuit board 20D, the second filler 70 is filled. Moreover, in a recessed portion surrounded by the wall portions 28-1, 28-2, 28-3, 28-4 also, the second filler 70 is filled.

A height h3 of the wall portions 28-1, 28-2, 28-3, 28-4 is such a height that a top surface of the electronic component 51 does not project from a back surface f4 of a circuit board 22D when the electronic component 51 is mounted in the mounting land 29, that is, the wall portions 28-1, 28-2, 28-3, 28-4 are formed in height higher than a height f4 of the electronic component 51. Moreover, the height h3 of the wall portions 28-1, 28-2, 28-3, 28-4 is preferable to be about 0.2 mm to 0.3 mm when the thickness of the circuit board 20D is 0.4 mm to 0.5 mm, that is, the height h3 of the wall portions 28-1, 28-2, 28-3, 28-4 is preferable to be about half the thickness of the circuit board 20D.

Furthermore, as for the size of the recessed portion surrounded by the wall portions 28-1, 28-2, 28-3, 28-4, it has such a length that the wall portions 28-1, 28-2, 28-3, 28-4 overlap with sensor electrodes 13 (bumps 14) in the vertical direction as shown in FIG. 13.

In the case of the semiconductor package in which the sensor electrodes 13 (bumps 14) are arranged in matrix, an influence of an outer periphery of the sensor electrodes 13 (bumps 14) and four corner portions of the sensor electrodes 13 (bumps 14) to warpage of the circuit board 20D is large; however, by forming the recessed portion in such a size that the wall portions 28-1, 28-2, 28-3, 28-4 overlap with the sensor electrodes 13 (bump 14) of the semiconductor package 10, the thickness of the circuit board 20D at the outer periphery of the sensor electrodes 13 (bumps 14) and at the four corner portions of the sensor electrodes 13 (bumps 14) can be made thick, and a warp of the circuit board 20D can thereby effectively reduced.

In the imaging unit 100D according to the fourth embodiment, the second filler 70 having a smaller linear expansion per unit length when heated from room temperature to sterilization temperature than that of the first filler 60 is filled on the joint surface between the semiconductor package 10 and the circuit board 20D, and around the joint portion of the electronic component 51 and the mounting land 29. Therefore, an influence of thermal expansion to the joint portion at the time of sterilization processing can be reduced. Moreover, by using a material highly resistant to moisture is used as the first filler 60, an influence of humidity to the semiconductor package 10 can be reduced.

Although the wall portions 28-1, 28-2, 28-3, 28-4 are formed on all four sides on the back surface of the circuit board 20D to mount the electronic component 51 in the recessed portion surrounded by the wall portions 28-1, 28-2, 28-3, 28-4 in the fourth embodiment, a warp of the circuit board 20D can be reduced as long as the wall portions 28-1, 28-2, 28-3, 28-4 are formed on at least two opposing sides.

An imaging unit and an endoscope of the disclosure are useful for endoscope systems for which high quality images and a reduced diameter at a distal end portion are needed.

According to some embodiments, a second filler with small linear expansion per unit length when heated from room temperature to sterilization temperature is used in a joint portion of a semiconductor package and a circuit board, thereby improving the reliability of the joint portion.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging unit comprising:
a semiconductor package that includes a light receiver of an imaging element formed on a front surface of the semiconductor package, and a sensor electrode formed on a back surface of the semiconductor package;
a circuit board that includes a connection electrode electrically and mechanically connected to the sensor electrode through a bump on a front surface of the circuit board;
an enclosing member configured to enclose the semiconductor package;
a first filler that is filled in space enclosed by the enclosing member; and
a second filler that is filled on a joint surface between the semiconductor package and the circuit board, and that has a smaller linear expansion per unit length at sterilization temperature than that of the first filler.

2. The imaging unit according to claim 1, wherein
a viscosity of the second filler before hardening is smaller than a viscosity of the first filler before hardening.

3. The imaging unit according to claim 1, wherein
the circuit board includes:
a first board in which a first connection electrode and a second connection electrode are formed on a front surface and a back surface of the first board, respectively, and in which the first connection electrode on the front surface of the first board is electrically and mechanically connected to the sensor electrode of the semiconductor package; and
a second board in which a third connection electrode is formed on a front surface of the second board and a cable connection electrode is formed on a side surface of the second board, and in which the third connection electrode is electrically and mechanically connected to the second connection electrode of the first board,
an electronic component is mounted in a recessed portion formed on the back surface of the first board or on the front surface of the second board, and
the first board, the second board, and a cable that is connected to the cable connection electrode fit within a size of a surface of projection of the semiconductor package in an optical axis direction of the semiconductor package when viewed along the optical axis direction of the semiconductor package.

4. The imaging unit according to claim 3, wherein
the second filler is filled at a joint portion between the first board and the second board, and in the recessed portion.

5. The imaging unit according to claim 1, wherein
the circuit board includes a main body in which the connection electrode is formed, and an attaching portion that protrudes from to a back surface of the main body and in which cable connection electrodes are formed on at least two opposing side surfaces out of protruding side surfaces of the attaching portion, and
the circuit board, and cables that are connected to the cable connection electrodes fit within a size of a surface of projection of the semiconductor package in an optical axis direction of the semiconductor package when viewed along the optical axis direction of the semiconductor package.

6. The imaging unit according to claim 5, wherein
an electronic-component mounting area in which a plurality of electronic components are mounted is arranged on a back surface of the main body,
the attaching portion protrudes from the main body such that a center plane of the two opposing side surfaces on which the cable connection electrodes are formed is shifted in position from a center plane of side surfaces of the semiconductor package parallel to two side surfaces of the attaching portion.

7. The imaging unit according to claim 6, wherein
the second filler is filled around a joint portion between the main body and the electronic components.

8. The imaging unit according to claim 1, wherein
the circuit board include: the connection electrode to which the sensor electrode is connected; a cable connection electrode to which a cable is connected; and a recessed portion in which an electronic component is mounted on a back surface of the circuit board, the connection electrode and the cable connection electrode being arranged side by side on the front surface of the circuit board, and
in the semiconductor package, the light receiver of the imaging element is arranged parallel to an optical axis direction.

9. The imaging unit according to claim 8, wherein
the second filler is filled around a joint portion between the circuit board and the electronic component.

10. An endoscope comprising
an insertion portion in which the imaging unit according to claim 1 is arranged at a distal end of the insertion portion.
